(19) United States
(12) Patent Application Publication
(10) Pub. No.: US 2009/0131630 A1
(43) Pub. Date: May 21, 2009

(54) Process For the Preparation of a Dicarboxylic Acid

(76) Inventors: Johannes Adrianus Maria Van Broekhoven, Amsterdam (NL); Eit Drent, Amsterdam (NL); Rene Ernst, Amsterdam (NL); Willem Wabe Jager, Amsterdam (NL); Cornelia Alida Krom, Amsterdam (NL)

Correspondence Address: SHELL OIL COMPANY, P O BOX 2463, HOUSTON, TX, 772522463, US

(21) Appl. No.: 11/884,081

(22) PCT Filed: Feb. 10, 2006
(86) PCT No.: PCT/EP06/50823

§ 371 Date: Oct. 23, 2007

(30) Foreign Application Priority Data
Feb. 11, 2005 (EP) 05101015.5

(51) Int. Cl. C07C 51/44; C08G 69/26

(52) U.S. Cl. 528/335; 562/521

(57) ABSTRACT

A process for the preparation of a dicarboxylic acid, comprising the steps of (a) contacting a conjugated diene with carbon monoxide and water in the presence of a catalyst system including a source of palladium, a source of an anion and a bidentate phosphine ligand, to obtain a mixture containing an ethylenically unsaturated acid and one or more reversible adduct of the conjugated diene and the ethylenically unsaturated acid; and (b) removing unreacted conjugated diene, and the reversible adducts of the conjugated diene from the reaction mixture; and (c) reacting the mixture obtained in step (b) containing the ethylenically unsaturated acid further with carbon monoxide and water to obtain the dicarboxylic acid.

### FIELD OF THE INVENTION

The present invention provides a process for the preparation of a dicarboxylic acid by carbonylation of a conjugated diene.

### BACKGROUND OF THE INVENTION

Carbonylation reactions of conjugated dienes are well known in the art. In this specification, the term carbonylation refers to a reaction of a conjugated diene under catalysis by a transition metal complex in the presence of carbon monoxide and water, as for instance described in WO 04/103948.

In WO 04/103948, a process is disclosed for the preparation of adipic acid from 1,3-butadiene or a mixture of 1,3-butadiene with olefinic products in a two-stage reaction. In the first stage of the disclosed process, 1,3-butadiene was reacted with carbon monoxide and water in the presence of a carbonylation catalyst comprising a palladium compound, a source of an anion and 1,2-bis(di-tert-butylphosphinomethyl)benzene as bidentate diphosphine ligand for several hours until substantially all of the 1,3-butadiene was converted. To the obtained mixture comprising pentenoic acid product and the catalyst, in the second step additional water and carbon monoxide were added and the reaction was continued until at least part of the pentenoic acid product was converted to adipic acid. It was found that the first reaction step, in spite of initially high catalyst activity, requires long reaction times, thereby making the overall process less suitable for industrial applicability.

Accordingly, there remained the need to provide for a process for the preparation of dicarboxylic acid with high turnover frequency in both carbonylation steps, thereby making the process suitable for industrial application.

It has now been found that the above identified process for the preparation of a dicarboxylic acid product from a conjugated diene can be very effectively performed as set out below, which makes it particularly suited as a semi-continuous or continuous industrial scale process.

### SUMMARY OF THE INVENTION

Accordingly, the subject invention provides a process for the preparation of a dicarboxylic acid, comprising the steps of
(a) contacting a conjugated diene with carbon monoxide and water in the presence of a catalyst system including a source of palladium, a source of an anion and a bidentate phosphine ligand, to obtain a mixture comprising an ethylenically unsaturated acid and one or more reversible adducts of the conjugated diene and the ethylenically unsaturated acid; and
(b) removing unreacted conjugated diene and the reversible adducts formed by the conjugated diene with the ethylenically unsaturated acid from the reaction mixture; and
(c) reacting the mixture obtained in step (b) further with carbon monoxide and water to obtain the dicarboxylic acid.

### FIGURES

FIG. 1 is a schematic representation of a preferred embodiment of the process according to the invention.

### DETAILED DESCRIPTION OF THE INVENTION

Applicants found that by partly converting the conjugated diene starting compound in step (a) and by separating non-converted conjugated diene and reversible adducts formed by the conjugated diene and the ethylenically unsaturated acid from the mixture comprising the catalyst system and the intermediate ethylenically unsaturated product, a very efficient process is obtained. By not allowing the reaction in step (a) to proceed to full conversion, long reaction times are avoided, which make the process less economical.

In contrast to the process disclosed in WO 04/103948, this permits to maintain high turn-over numbers throughout the process, since the reaction to full conversion of the butadiene becomes very slow in particular towards the end of the reaction when most butadiene has been converted. The removal of conjugated diene and its reversible diene adducts has the additional advantage that the obtained mixture comprising the catalyst can be directly subjected to the second reaction step (c), without necessitating a long induction period until the carbonylation of the ethylenically unsaturated acid ensues.

A further advantage resides in the fact that the catalyst exposure to high temperatures can be reduced, thereby increasing catalyst lifetime. Furthermore, less Diels-Alder by-products are formed from the ethylenically unsaturated acid and the conjugated diene, which are non-reversible under the conditions used for the carbonylation reaction, and hence reduce the overall yield.

The subject process is based on the insight that the catalyst system hardly converts any of the obtained pentenoic acid products before the conjugated diene present in the reaction mixture is fully converted, in spite of the fact that the catalyst system is in principle capable of converting the ethylenically unsaturated product with good reactivity.

Yet a further advantage of the subject process resides in the fact that the high selectivity for conjugated diene reactants in the first step of the process has the advantage that the feed containing the conjugated diene reactant does not necessarily have to be free of alkenes or even alkynes. Even an admixture with up to 55 mol % of alkenes and/or alkynes based on the diene reactant was tolerated in the feed without significant carbonylation of these alkenes or alkynes.

It was found that conjugated dienes have the tendency to reversibly form allylic alkenyl esters with any carboxylic acid present in the reaction mixture, in particular under catalysis by the carbonylation catalyst. Depending on the reaction conditions, these alkenyl esters can be formed in substantial amounts.

Without wishing to be bound to any particular theory, it is believed that the formation of the esters from the conjugated diene and the ethylenically unsaturated acid product is an equilibrium reaction catalyzed by the carbonylation catalyst, albeit at a comparatively slow rate. The presence of a high concentration of the conjugated diene, as well as an increasing amount of carboxylic acids with suitable reactivity favors the formation of esters. In absence of catalyst, the equilibrium reaction becomes very slow, hence effectively freezing the equilibrium. Without wishing to be bound to any particular theory, it is believed that this is due to the presence of reversible diene adducts, which only slowly revert back to the conjugated diene and the acid to which they stand in equilibrium, even under catalysis by the palladium carbonylation catalyst. Accordingly, the overall reaction rate becomes increasingly dependent on speed of the reversion of the reversible esters to conjugated diene.

Since the alkenyl esters can be reverted into the conjugated diene and the ethylenically unsaturated acid, they are referred to as “reversible diene adducts” throughout the present specification. These “reversible diene adducts” were found to be remarkably stable in absence of the carbonylation catalyst.

In the case of 1,3-butadiene as conjugated diene, the “reversible diene adducts” are butenyl esters with any carboxylic acid present in the reaction mixture, thus mainly butyl-esters of 2-, 3- and 4-pentenoic acid, and mixtures thereof. In the case of 1,3-butadiene as conjugated diene, the term ethylenically unsaturated acid product describes 2-pentenoic acid, 3-pentenoic acid and 4-pentenoic acid, and mixtures thereof.

In order to avoid arriving at a low concentration of conjugated diene, step (a) of the present process is therefore not allowed to proceed to full conversion of the conjugated diene and its reversible adducts, but only to partial conversion. Then unreacted conjugated diene and the reversible diene adducts are removed from the reaction mixture in step (b).

In the case of the carbonylation of 1,3-butadiene, step (a) is preferably allowed to proceed to 95% of conversion, based on moles of 1,3-butadiene converted versus moles of 1,3-butadiene fed. Yet more preferably, step (a) is allowed to proceed to 85% of conversion, again more preferably to 75% of conversion, again more preferably step to 65% of conversion, and most preferably step (a) is allowed to proceed to 60% of conversion. Again more preferably, the reaction is conducted in such way, the conversion of 1,3-butadiene is in step (a) in the range of from 30 to 60%, based on moles of 1,3-butadiene converted versus moles of 1,3-butadiene fed.

In step (a), the ratio (v/v) of conjugated diene and water in the feed can vary between wide limits and suitably lies in the range of 1:0.0001 to 1:500. However, it was found that the addition of water in step (a) to the reaction medium in order to provide a higher concentration of the reactant and hence an increased reaction rate had the opposite effect, i.e. an increase of the water concentration resulted in a strongly decreased reaction rate. Therefore, preferably, in step (a), less than 5% by weight of water is present in the reactor, yet more preferably, less than 3% by weight of water, yet more preferably, less than 1% by weight of water, again more preferably less than 0.15% by weight of water, and most preferably less than 0.001% by weight of water (w/w) is present in the reactor, calculated on the total weight of reactants. Again more preferably, these water concentrations are continuously present only, in particular if the reaction is performed as semi-batch or as continuous process. The water concentration may be determined by any suitable method, for instance by a Karl-Fischer-titration.

It was equally found that the polarity of the reaction mixture influences the reaction speed, i.e. the reaction of step (a) is favored by a more apolar medium. This may be achieved for instance by addition of an apolar solvent e.g. toluene. It was also found that if the diene feed contained alkenes and alkynes, since the amount of these apolar compounds was higher in the reaction medium at a constant level of conjugated diene, the overall medium was le\dot{s} polar, and the reaction equally proceeded faster.

The reaction rate towards the end of the reaction can be somewhat increased by increased temperature, this however reduces the catalyst lifetime.

According to the present process, the conjugated diene and reversible diene adducts are removed in process step (b) from the reaction medium obtained in step (a) to avoid the slowing down of the reaction rate when a high degree of diene conversion is approached. Thereby, carbon monoxide, conjugated diene and the reversible ester products are removed from the reactor, while at least part of the ethylenically unsaturated acid and the catalyst system remain in the reactor.

According to a preferred embodiment of the process, the unreacted conjugated diene and the reversible diene adducts are removed from the reaction mixture obtained in step (a) by first releasing the pressure of the system to near atmospheric pressure, thereby releasing the carbon monoxide, and subsequently the unreacted conjugated diene and its reversible adducts are removed. The latter may be removed from the reaction mixture by an in-situ conversion and simultaneous removal of the conjugated diene, or removed as such, and either recycled to step (a) or reversed into the educts first in a separate reaction step, before the products are recycled or forwarded to the appropriate reaction stage.

The in-situ conversion is preferably done in the following manner: provided the conjugated diene is gaseous or has a low boiling point at ambient pressure, as for instance the case of 1,3-butadiene, the reaction mixture obtained in step (a) is brought near to atmospheric pressure, and then the conjugated butadiene is stripped from the reaction mixture under a gas flow, the gas flow preferably comprising carbon monoxide to provide additional stability to the catalyst. In this way, the reversible diene adducts are forced to revert back into the conjugated diene and the ethylenically unsaturated acid, since constant removal of the conjugated diene with the gas stream will move the equilibrium towards reversion. The gaseous stream obtained in the stripping comprising carbon monoxide and conjugated diene may then advantageously be returned to step (a).

Alternatively, the reversible diene adducts may be removed from the reaction mixture in a distillative operation. The removed obtained ester mixture, usually also comprising some ethylenically unsaturated acid and by-products, is then either directly recycled to step (a), or converted in a separate conversion step in the presence of a suitable catalyst into conjugated diene and ethylenically unsaturated compound. At this point in the process, other undesired side-products, such as the Diels-Alder products or polymeric conjugated diene may preferably be removed as well. The Diels-Adler products of the conjugated diene and the ethylenically unsaturated acid are preferably removed from the mixture removed in step (b) in a distillate operation.

For conversion, the reversible diene adducts are contacted with a suitable catalyst before recycling the obtained conjugated diene and the unsaturated acid back to the process. Any catalyst suitable for the conversion may be applied, such as heterogeneous or homogeneous palladium catalysts, or acidic heterogeneous catalysts. An example of a suitable palladium catalyst is the catalyst system as described for step (a) and (c).

The reversible diene adducts usually have a boiling range below that of the unsaturated acid product. If 1,3-butadiene is the conjugated diene, the distillative removal is preferably performed at a bottom temperature in range of from 70 to 150° C. and a pressure of from 1 to 30 kPa (10 to 300 mbar), yet more preferably at a bottom temperature in range of from 90 to 130° C. and a pressure of from 2.5 to 15 kPa, and most preferably, at a bottom temperature in the range of from 100 to 110° C. and at a pressure in the range of from 3 to 8 kPa. Although these pressures and temperatures are not critical, pressures of above 20 kPa should be avoided due to the high temperatures required, which may result in catalyst degradation, while pressures below 1 kPa will require specific equipment. Although the removal of the conjugated diene and its reversible diene adducts by distillation is a more complex process than the process employing the in-situ conversion, the carbonylation catalyst is used more effectively.

The subject process permits to react conjugated dienes with carbon monoxide and a co-reactant. The conjugated diene reactant has at least 4 carbon atoms. Preferably the diene has from 4 to 20 and more preferably from 4 to 14 carbon atoms. However, in a different preferred embodiment, the process may also be applied to molecules that contain conjugated double bonds within their molecular structure, for instance within the chain of a polymer such as a synthetic rubber. The conjugated diene can be substituted or non-substituted. Preferably the conjugated diene is a non-substituted diene. Examples of useful conjugated dienes are 1,3-butadiene, conjugated pentadienes, conjugated hexadienes, cyclopentadiene and cyclohexadiene, all of which may be substituted. Of particular commercial interest are 1,3-butadiene and 2-methyl-1,3-butadiene (isoprene).

In step (c), the mixture obtained in step (b) is pressurized again with carbon monoxide, and additional water is added as reactant for the carbonylation. Herein, the ethylenically unsaturated acid formed in step (a) is converted to a dicarboxylic acid under addition of carbon monoxide and water.

It was found that the reaction of the formed ethylenically unsaturated carboxylic acid to a diacid proceeds at an increased rate if the polarity of the medium is increased with respect to step (a). Therefore preferably, the water concentration throughout step (c) is higher as compared to step (a). Accordingly, the present invention relates to a process wherein in step (c) the water concentration in the reaction medium is maintained within the range of from to 1 to 50%, preferably from 2 to 30%, more preferably from 3 to 25%, and most preferably from 5 to 10% (w/w), based on the amount of the total liquid reaction medium. Preferably, step (c) is performed as semi-batch or as continuous process, and more preferably, all of steps (a), (b) and (c) are performed continuously. Again, more preferably, the process is performed in such way, that step (a) is performed at a water concentration of less than 0.1% (w/w), based on the amount of the total liquid reaction medium, while step (c) is performed at a water concentration of above 3% (w/w), based on the amount of the total liquid reaction medium.

In the case of the carbonylation of 1,3-butadiene, step (c) results in adipic acid product and in high purity. Adipic acid is a highly crystalline solid at ambient conditions. In the case that the process is conducted in pentenoic acid as solvent, adipic acid may begin to crystallize from the reaction mixture from a certain concentration and temperature onwards. If spontaneous crystallization in the reactor for step (c) is not desired, preferably, step (c) is only allowed to proceed until the liquid reaction medium comprises a saturated solution of adipic acid and/or any by-products at the reaction temperature in the liquid reaction medium.

Suitable sources of palladium for steps (a) and (c) include palladium metal and complexes and compounds thereof such as palladium salts; and palladium complexes, e.g. with carbon monoxide or acetyl acetonate, or palladium combined with a solid material such as an ion exchanger. Preferably, a salt of palladium and a carboxylic acid is used, suitably a carboxylic acid with up to 12 carbon atoms, such as salts of acetic acid, propionic acid and butanoic acid, or salts of substituted carboxylic acids such as trichloroacetic acid and trifluoroacetic acid. A very suitable source is palladium (II) acetate.

Any bidentate diphosphine resulting in the formation of an active carbonylation catalyst with palladium may be used in the subject process. Preferably, a bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom due to the higher activity and/or selectivity found with such catalysts in both reaction steps. Yet more preferably, R represents an aromatic bidentate bridging group that is substituted by one or more alkylene groups, and wherein the phosphino groups R¹R²P— and —PR³R⁴ are bound to the aromatic group or to the alkylene group due to the observed high stability of these ligands. Most preferably R¹, R², R³ and R⁴ are chosen in such way, that the phosphino group PR¹R² differs from the phosphino group PR³R⁴. A very suitable ligand is 1,2-bis(di-tert-butylphosphinomethyl)benzene.

The ratio of moles of a bidentate diphosphine per mole atom of palladium preferably ranges from 0.5 to 50, more preferably from 0.8 to 10, yet more preferably from 0.9 to 5, yet more preferably in the range of 0.95 to 3, again more preferably in the range of 1 to 2, and yet most preferably it is stoichiometric. In the presence of oxygen, slightly higher than stoichiometric amounts of ligand to palladium are beneficial.

The source of anions preferably is an acid, more preferably a carboxylic acid, which preferably serves both as catalyst component as well as solvent for the reaction. Again more preferably, the source of anions is an acid having a pKa above 2.0 (measured in aqueous solution at 18° C.), and yet more preferably an acid having a pKa above 3.0, and yet more preferably a pKa of above 3.6. Examples of preferred acids include carboxylic acids, such as acetic acid, propionic acid, butyric acid, pentanoic acid, pentenoic acid and nonanoic acid, the latter three being highly preferred as their low polarity and high pKa was found to increase the reactivity of the catalyst system. 2- and/or 3-Pentenoic acid is particularly preferred in case the conjugated diene is 1,3-butadiene. Preferably the reaction is conducted in 2-pentenoic acid, 3-pentenoic acid and/or 4-pentenoic acid, since this was found to not only form a highly active catalyst system, but also to be a good solvent for all reaction components.

The molar ratio of the source of anions, and palladium is not critical. However, it suitably is between 2:1 and 10⁹:1 and more preferably between 10⁷:1 and 10:1, yet more preferably between 10⁶:1 and 10²:1, and most preferably between 10⁵:1 and 10²:1 due to the enhanced activity of the catalyst system. Very conveniently the acid corresponding to the desired product of the reaction can be used as the source of anions in the catalyst. The process may optionally be carried out in the presence of an additional solvent, however preferably the intermediate acid product serves both as source of anions and as reaction solvent. Usually amounts in the range of 10⁻⁸ to 10⁻¹, preferably in the range of 10⁻⁷ to 10⁻² mole atom of palladium per mole of conjugated diene are used, preferably in the range of 10⁻⁵ to 10⁻² mole atom per mole of conjugated diene. If the amount of catalyst is chosen at a level below 20 ppm, calculated on the total amount of liquid reaction medium, side reactions, in particular Diels-Alder reactions of the conjugated diene, become more prominent. In the case of 1,3-butadiene, side-products formed include 4-vinyl cyclohexene (further referred to as VCH, being the adduct of two 1,3-butadiene molecules), and 2-ethyl cyclohexene carboxylic acid, further referred to as ECCA, which is the adduct of 1,3-butadiene and 2-pentenoic acid. The formation of ECCA is favoured if pentenoic acid also serves a solvent. When about 20 ppm of palladium catalyst were employed, ECCA was formed in up to 3% by weight on total products formed. An increase of the catalyst concentration to 200 ppm is expected to result in a reduction of to 0.3% by weight of ECCA, and an increase of the catalyst concentration to 1000 ppm is expected to result in a reduction to 0.06% by weight. Accordingly, in steps (a) and (b), the carbonylation is preferably performed in the presence of at least 20 ppm of catalyst, more preferably in the presence of 100 ppm of catalyst, and most preferably in the presence of at least 500 ppm. Although this requires a larger amount of palladium to be employed, the catalyst may advantageously be recycled to the reaction of either step (a) or (b). Examples of suitable catalyst systems as described above are those disclosed in EP-A-1282629, EP-A-1163202, WO2004/103948 and/or WO2004/103942. Most preferably, the reaction is performed in the ethylenically unsaturated acid and/or the dicarboxylic acid product, provided the mixture remains liquid at reaction conditions.

The carbonylation reaction according to the present invention in steps (a) and (c) is carried out at moderate temperatures and pressures. Suitable reaction temperatures are in the range of 0-250° C., more preferably in the range of 50-200° C., yet more preferably in the range of from 80-150° C.

The reaction pressure is usually at least atmospheric pressure. Suitable pressures are in the range of 0.1 to 25 MPa (1 to 250 bar), preferably in the range of 0.5 to 15 MPa (5 to 150 bar), again more preferably in the range of 0.5 to 9.5 MPa (5 to 95 bar) since this allows use of standard equipment. Carbon monoxide partial pressures in the range of 1 to 9 MPa (10 to 90 bar) are preferred, the upper range of 5 to 9 MPa being more preferred. Again higher pressures require special equipment provisions, although the reaction would be faster since it was found to be first order with carbon monoxide pressure.

In the process according to the present invention, the carbon monoxide can be used in its pure form or diluted with an inert gas such as nitrogen, carbon dioxide or noble gases such as argon, or co-reactant gases such as ammonia.

Process steps (a) to (c) are preferably performed in a continuous operation. Steps (a) and (c) of the subject process are suitably performed in a single reactor suitable for gas-liquid reactions, or a cascade thereof, such as constant flow stirred tank reactor, or a bubble column type reactor, as for instance described in “Bubble Column Reactors” by Wolf-Dieter Deckwer, Wiley, 1992. A bubble column reactor is a mass transfer and reaction device in which in one or more gases are brought into contact and react with the liquid phase itself or with a components dissolved or suspended therein. Preferably, a reactor with forced circulation is employed, which is generally termed an “ejector reactor”, or if the reaction medium is recycled to the reactor, “ejector loop reactor”. Such reactors are for instance described in U.S. Pat. No. 5,159,092 and JP-A-11269110, which employ a liquid jet of the liquid reaction medium as a means of gas distribution and circulation.

The dicarboxylic acid may be isolated from the reaction mixture by various measures. Preferably, the dicarboxylic acid is isolated from the reaction mixture by crystallization of the diacid in the reaction mixture and separation of the diacid crystals from the remaining reaction mixture containing the catalyst. It has been found that the diacid crystals can be obtained in a high purity in only a few crystallization steps, making it an efficient method for the separation of the product from the catalyst and unreacted ethylenically unsaturated acid intermediate. The subject process further preferably comprises a further process step (e) of purifying the dicarboxylic acid obtained in step (d). The process further preferably comprises the steps of (f) converting the dicarboxylic acid to its dichloride, and (g) reacting the dicarboxylic acid dichloride with a diamine compound to obtain an alternating co-oligomer or co-polymer.

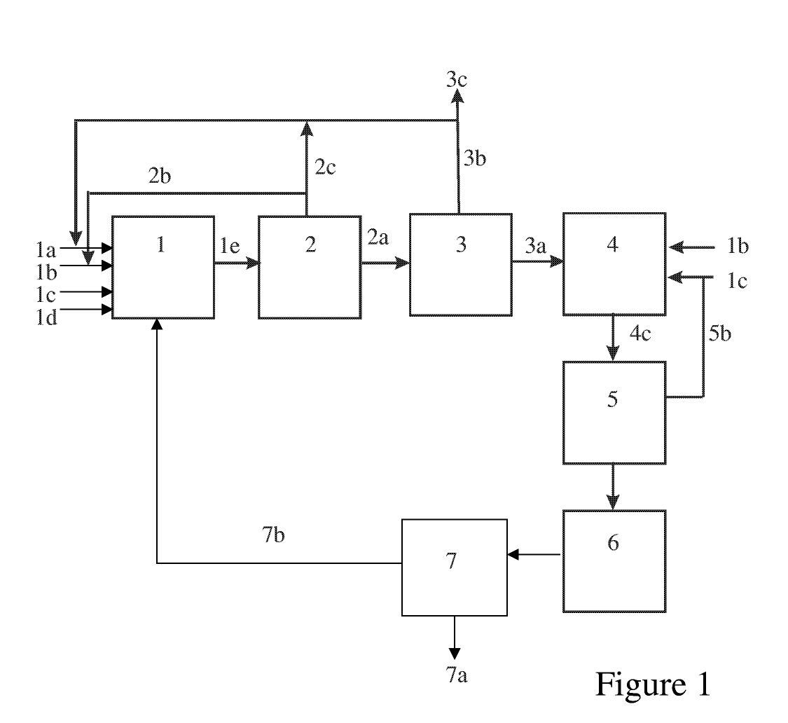

The invention will further be described by way of example with reference to FIG. 1.

FIG. 1 is a schematic representation of a preferred embodiment of the process according to the present invention. FIG. 1 illustrates a process wherein a conjugated diene (1a), carbon monoxide (1b), water (1c) and a catalyst system including a source of palladium, a source of an anion and a bidentate phosphine ligand (1d) are supplied to a reactor (1). In this reactor (1), the conjugated diene is contacted with the carbon monoxide and water in the presence of a catalyst system including a source of palladium, a source of an anion and a bidentate phosphine ligand, to obtain a mixture comprising an ethylenically unsaturated acid product (1e). The mixture (1e) is then transported to vessel (2), where it is depressurized to obtain a depressurized mixture (2a). At this stage, optionally a stream of a normally gaseous conjugated diene (2c) and a stream of unreacted carbon monoxide (2b) may be separated from the mixture (1e). These may be recycled to reactor (1). The depressurized mixture (2a) is then transported into a vessel (3), wherein it is converted in-situ back into the conjugated diene and into the ethylenically unsaturated acid. A stream (3b) comprising conjugated diene is removed to obtain a mixture (3a) comprising the ethylenically unsaturated acid product together with the catalyst system.

The stream (3b) comprising conjugated diene is then recycled to the reactor (1), optionally in admixture with stream 2c.

The obtained depressurized mixture (3a) free from conjugated diene and reversible adducts thereof is transferred to a reactor (4), where it is reacted further under carbon monoxide pressure (1b) with additional water (1c) to obtain a stream (4c) comprising the dicarboxylic acid in admixture with the ethylenically unsaturated acid and the catalyst system. The stream 4c is then depressurized (5), while remaining carbon monoxide (5b) is recycled to step (4). The depressurized mixture 5a is then cooled (6), and subjected to filtration (7) of the obtained crystals of the dicarboxylic acid, yielding crude adipic acid crystals (7a) and a liquid filtrate (7b). The liquid filtrate (7b) comprising the catalyst system in admixture with the ethylenically unsaturated acid is then recycled to step (1).

The invention will be illustrated by the following, non-limiting example:

### EXAMPLE 1

### Semi Continuous Reaction for Producing Adipic Acid from Butadiene

A 1.2 l mechanically stirred autoclave was charged with 130 g pentenoic acid and 10 g tetradecane. The autoclave was degassed three times with carbon monoxide at 3.0 MPa. Next the autoclave was pressurised with carbon monoxide to a pressure of 5.0 MPa. Then, 25 g of 1,3-butadiene were pumped intro the reactor. Next a solution of 0.2 mmol of palladium acetate and 0.4 mmol of 1,2-bis(di-tert-butylphosphinomethyl)benzene dissolved in 10 g pentenoic acid was injected into the reactor. The injector was rinsed with a further 10 g of pentenoic acid. Then butadiene and water were continuously added to the reactor at a rate of 40 mmol/h, while the reactor was heated to 105° C. over a period of 30 minutes. When this temperature has been reached the pressure was adjusted to 8.0 MPa, and these conditions were maintained for about 120 hours, and the reaction was monitored by taking samples of the reaction mixture at regular intervals. Once a TON of 20,000 mol pentenoic acid/mol catalyst was achieved, the feed of butadiene and water was stopped. At the end of this period, the water concentration corresponded to less than 0.1% w/w, calculated on the total amount of reaction components in the reactor.

Then pressure was released to ambient pressure, and carbon monoxide was bubbled through the reactor at atmospheric pressure for approximately 25 hours. After this period, no 1,3-butadiene or reversible diene adducts (esters of 1,3-butadiene with pentenoic acids) could be detected in the reaction mixture.

Then water was added until the water concentration was about 7% w/w, calculated on the total amount of reaction components in the reactor, and the reactor was heated to 105° C. and pressurized to 8.0 MPa with carbon monoxide. The water concentration was maintained at approximately 7% w/w of the reactor mixture for 52 hours, then the continuous water addition was stopped. The reaction was continued for a further 63 hours, when approximately 20% w/w of the reactor mixture consisted of adipic acid, at a final water concentration was 0.1% w/w, calculated on the total amount of reaction components in the reactor. The adipic acid was obtained with an overall selectivity starting from butadiene of 94%.

The adipic acid product was filtrated off, and the remaining liquid phase comprising the catalyst system in admixture with pentenoic acid showed a similar activity and selectivity for the carbonylation of 1,3-butadiene.

The example shows that the removal of reversible esters, preferably in combination of a low water concentration in the first reaction step, and a high water concentration in the second step allows to obtain adipic acid in high purity and with an overall high turn over frequency. Moreover, a single catalyst system can be employed, which can be easily recycled over the process. This makes the present process suitable for a continuous industrial process.

1. A process for the preparation of a dicarboxylic acid, comprising the steps of
(a) contacting a conjugated diene with carbon monoxide and water in the presence of a catalyst system including a source of palladium, a source of an anion and a bidentate phosphine ligand, to obtain a mixture containing an ethylenically unsaturated acid and one or more reversible adduct of the conjugated diene and the ethylenically unsaturated acid; and
(b) removing unreacted conjugated diene and the reversible adducts formed by the conjugated diene with the ethylenically unsaturated acid from the reaction mixture; and
(c) reacting the mixture obtained in step (b) further with carbon monoxide and water to obtain the dicarboxylic acid product.

2. The process of claim 1, wherein in step (a) the water concentration is maintained at a range of from 0.001 to less than 3% by weight of water, calculated on the overall weight of the liquid reaction medium.

3. The process of claim 1, wherein in step (c) the water concentration is maintained at a range of from 1% to 50% by weight of water, calculated on the overall weight of the liquid reaction medium.

4. The process of claim 1, further comprising a step (d) of separating the dicarboxylic acid from the reaction mixture obtained in step (c) to obtain a fraction comprising at least part of the catalyst, and recycling of the fraction comprising at least part of the catalyst obtained in step (d) to step (a).

5. The process of claim 1, wherein the removal in step (b) comprises the steps
(i) of bringing the reaction mixture obtained in step (a) to near atmospheric pressure, and
(ii) stripping the conjugated butadiene from the reaction mixture.

6. The process of claim 1, wherein the removal step (b) comprises removing the reversible adducts of the conjugated diene and the ethylenically unsaturated acid from the reaction mixture in a distillate operation.

7. The process of claim 6, wherein the removed reversible adducts of the conjugated diene and the ethylenically unsaturated acid are subsequently converted to the conjugated diene and the ethylenically unsaturated acid in the presence of a suitable catalyst.

8. The process of claim 6, wherein the removed reversible adducts of the conjugated diene and the ethylenically unsaturated acid are recycled to step (a).

9. The process claim 6, wherein Diels-Adler products of the conjugated diene and the ethylenically unsaturated acid are removed from the mixture removed in step (b) in a distillate operation.

10. The process of claim 1, wherein the conjugated diene is recycled from removal step (b) to step (a).

11. The process of claim 1, wherein the conjugated diene is 1,3-butadiene.

12. The process of claim 1, wherein the ethylenically unsaturated acid of step (a) is employed as solvent for the process.

13. The process of claim 1, wherein the bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom.

14. The process of claim 13, wherein R represents an aromatic bidentate bridging group that is substituted by one or more alkylene groups, and wherein the phosphino groups R¹R²P— and —PR³R⁴ are bound to the aromatic group or to the alkylene group.

15. The process of claim 13, wherein R¹, R², R³ and R⁴ are chosen in such way, that the phosphino group PR¹R² differs from the phosphino group PR³R⁴.

16. The process of claim 1, wherein steps (a), (b) and (c) are performed continuously.

17. The process of claim 1, wherein the catalyst system is present in an amount of at least 20 ppm, calculated on the total amount of liquid reaction medium.

18. The process of claim 1, further comprising a step (e) of purifying the dicarboxylic acid obtained in step (d).

19. The process of claim 18, further comprising the steps of
(f) converting the dicarboxylic acid to its dichloride, and
(g) reacting the dicarboxylic acid dichloride with a diamine compound to obtain an alternating co-oligomer or co-polymer.
